(19) United States
(12) Patent Application Publication
(10) Pub. No.: US 2023/0047257 A1
(43) Pub. Date: Feb. 16, 2023

(54) CADENCE MEASUREMENT DEVICE AND CADENCE MEASUREMENT METHOD

(71) Applicant: Alps Alpine Co., Ltd., Tokyo (JP)

(72) Inventors: Takayuki Ito, Miyagi-ken (JP); Jouichi Itoh, Miyagi-ken (JP)

(21) Appl. No.: 17/974,167

(22) Filed: Oct. 26, 2022

Related U.S. Application Data
(63) Continuation of application No. PCT/JP2021/008180, filed on Mar. 3, 2021.

(30) Foreign Application Priority Data
May 8, 2020 (JP) 2020-082718

(51) Int. Cl. A63B 24/00

(52) U.S. Cl. CPC A63B 24/0062; A63B 2220/836; A63B 2220/17

(57) ABSTRACT

A cadence measurement device includes a myoelectric potential sensor attachable to a leg portion of a living body, a waveform processor that performs waveform processing to obtain a myoelectric potential waveform from an output signal from the myoelectric potential sensor, and a cadence deriving unit that derives cadence according to the myoelectric potential waveform obtained by the waveform processing.

### CLAIM OF PRIORITY

This application is a Continuation of International Application No. PCT/JP2021/008180 filed on Mar. 3, 2021, which claims benefit of Japanese Patent Application No. 2020-082718 filed on May 8, 2020. The entire contents of each application noted above are hereby incorporated by reference.

### BACKGROUND

### 1. Field of the Disclosure

The present disclosure relates to a cadence measurement device and a cadence measurement method.

### 2. Description of the Related Art

In a conventional bicycle-type exercise instrument, a pedaling measurement unit attached to the bicycle-type exercise instrument has a rotational speed sensor. The bicycle-type exercise instrument measures cadence, which is the rotational speed of a crank portion, according to an output signal from the rotational speed sensor (see Japanese Unexamined Patent Application Publication No. 2018-110730, for example).

The use by conventional bicycle-type exercise instrument of a rotational speed sensor to measure cadence complicates the structure of the bicycle-type exercise instrument.

### SUMMARY

The present invention provides a cadence measurement device having a simple structure and also provides a cadence measurement method.

In one embodiment, the cadence measurement device includes a myoelectric potential sensor attached to a leg portion of a living body, a waveform processing unit that performs waveform processing to obtain a myoelectric potential waveform from an output signal from the myoelectric potential sensor, and a cadence deriving unit that derives cadence according to the myoelectric potential waveform obtained by the waveform processing.

The present invention can provide a cadence measurement device having a simple structure and can also provide a cadence measurement method.

### BRIEF DESCRIPTION OF THE DRAWINGS

FIG. 1 illustrates the structure of a cadence measurement device in an embodiment;

FIGS. 2A and 2B are drawings used to explain waveform processing executed by a waveform processing unit;

FIG. 3 is a drawing used to explain waveform processing executed by the waveform processing unit:

FIG. 4 is a drawing used to explain waveform processing executed by the waveform processing unit;

FIG. 5 is a drawing used to explain waveform processing executed by the waveform processing unit;

FIG. 6 is a drawing used to explain processing executed by a cadence deriving unit.

FIG. 7 illustrates table data that relates cadence and the period of one cycle to each other; and

FIG. 8 is a flowchart illustrating processing executed by the cadence measurement device.

### DESCRIPTION OF THE EXEMPLARY EMBODIMENTS

Embodiments to which a cadence measurement device and a cadence measurement method in the present invention are applied will be described below.

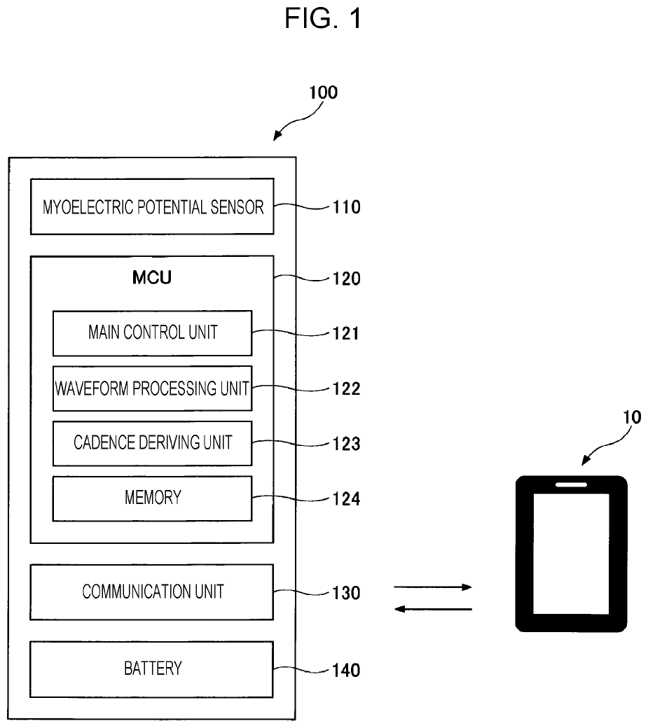

FIG. 1 illustrates an embodiment of a structure of a cadence measurement device 100. In FIG. 1, a smart phone 1 is also illustrated. The cadence measurement device 100 in FIG. 1 is illustrated larger than the smart phone 10 to illustrate the structure of the cadence measurement device 100 in detail. In actuality, however, the cadence measurement device 100 is smaller and more lightweight than the smart phone 10.

The cadence measurement device 100 includes a myoelectric potential sensor 110, a micro control unit (MCU) 120, a communication unit 130, and a battery 140. As an example, the cadence measurement device 100 is attached to the thigh of the user who pedals a bicycle or bicycle-type exercise instrument by being stuck with, for example, a tape or the like. Then, the cadence measurement device 100 measures cadence. Cadence is a number of revolutions of the crank axis of the pedal per minute. The cadence measurement device 100 only needs to measure the myoelectric potential of the surface of the leg portion of the user with the myoelectric potential sensor 110. As an example, the cadence measurement device 100 is stuck so as to measure the myoelectric potential of the quadriceps muscle.

The myoelectric potential sensor 110 has an analog amplifier circuit and a plurality of bioelectrodes stuck to the thigh of the user. The myoelectric potential sensor 110 amplifies a waveform signal, which represents a myoelectric potential detected in a hyperbolic lead method by using the plurality of bioelectrodes, with the analog amplifier circuit, and then outputs the amplified waveform signal. The output of the myoelectric potential sensor 110, which is an example of an output signal, is input into the MCU 120.

The MCU 120 has a main control unit 121, a waveform processing unit 122, a cadence deriving unit 123, and a memory 124. The MCU 120 is implemented by a microcomputer that includes a central processing unit (CPU), a random-access memory (RAM), a read-only memory (ROM), and the like. The main control unit 121, waveform processing unit 122, cadence deriving unit 123 represent the functions of programs executed by the MCU 120 as functional blocks. The memory 124 also functionally represents a memory in the MCU 120.

The main control unit 121, which is a processing unit that controls the MCU 120 in a centralized manner, executes processing other than processing executed by the waveform processing unit 122 and cadence deriving unit 123. Specifically, the main control unit 121 performs analog-to-digital (A/D) conversion processing on the output signal from the myoelectric potential sensor 110 to convert the output signal to a digital waveform signal, and performs other processing, for example.

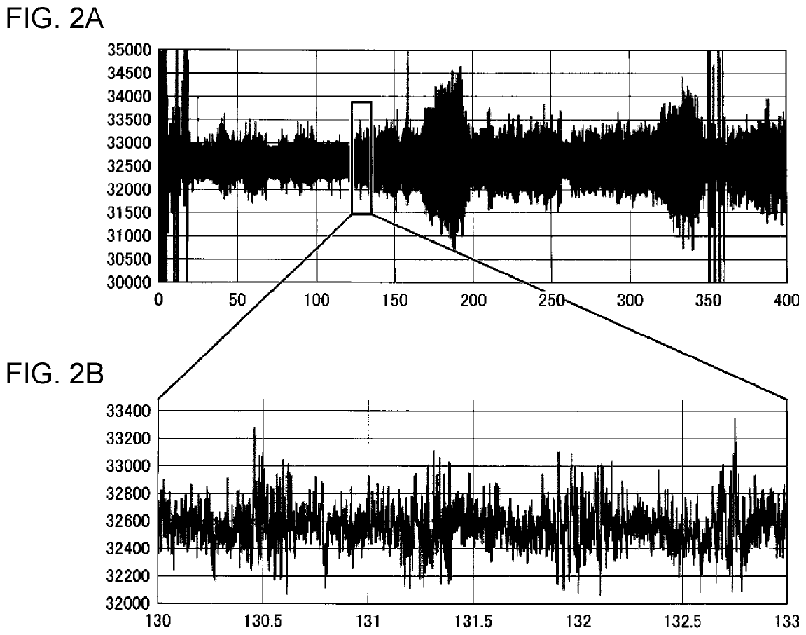

The waveform processing unit 122 acquires data representing a myoelectric potential waveform by performing waveform processing on the output signal from the myoelectric potential sensor 110. Waveform processing performed by the waveform processing unit 122 will be described later with reference to FIGS. 2A and 2B to FIG. 5.

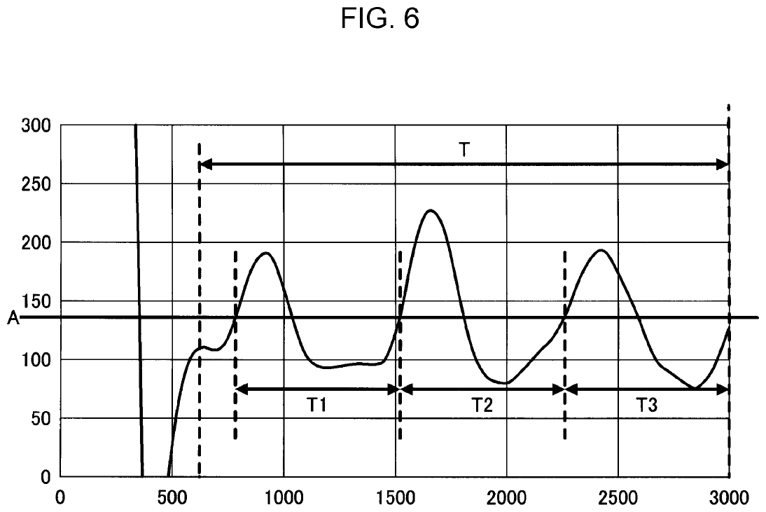

The cadence deriving unit 123 derives cadence from the myoelectric potential waveform acquired by the waveform processing unit 122. Waveform processing performed by the cadence deriving unit 123 will be described later with reference to FIG. 6.

The memory 124, which is an example of a storage unit, stores programs and data needed by the MCU 120 to perform processing as well as other data, and also temporarily stores data to be used by the main control unit 121, waveform processing unit 122, and cadence deriving unit 123 in processing and for other purposes.

The communication unit 130, which is a communication unit in Bluetooth Low Energy (BLE) (registered trademark) as an example, performs data communication with the smart phone 10. Measurement results from the cadence measurement device 100 are transmitted to the smart phone 10 through the communication unit 130. Application programs for use for the cadence measurement device 100 are installed in the smart phone 10, as an example. The smart phone 10 displays or otherwise processes data that an application program has received from the cadence measurement device 100 on the display or the like of the smart phone 10.

The battery 140 is a power source that supplies electric power to the myoelectric potential sensor 110, MCU 120, and communication unit 130. The battery 140 only needs to be a secondary battery, as an example. The battery 140 can be charged regardless of whether the battery 140 is incorporated into the cadence measurement device 100 or is removed from the cadence measurement device 100.

FIGS. 2A and 2B to FIG. 5 are drawings used to explain waveform processing executed by the waveform processing unit 122. In FIGS. 2A and 2B to FIG. 5, the horizontal axis indicates time and the vertical axis indicates amplitude. FIG. 2A illustrates the waveform of a digital waveform signal created by the main control unit 121 through A/D conversion. FIG. 2B illustrates part (segment for three seconds) of the waveform in FIG. 2A by enlarging the part along the horizontal-axis. The digital waveform signal illustrated in FIGS. 2A and 2B represents a myoelectric potential by electromyo-graphy (EMG) but includes noise and the like as well.

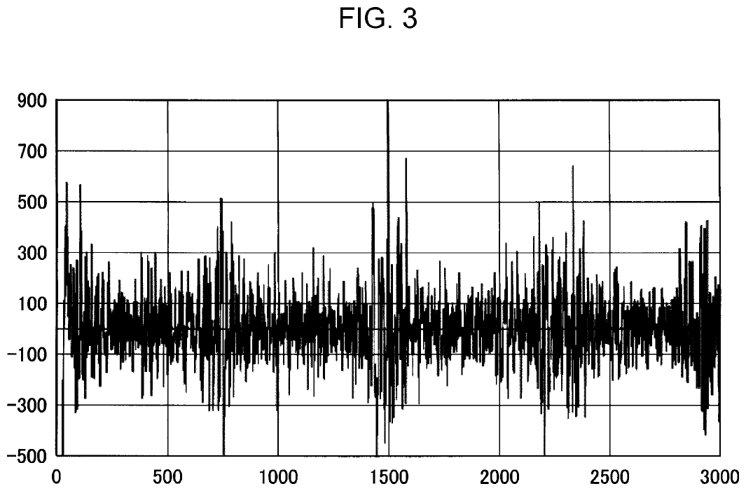

FIG. 3 illustrates a component waveform after the waveform processing unit 122 has performed band-pass filter processing on the digital waveform signal illustrated in FIG. 2B. Band-pass filter processing is filter processing (waveform processing) to pass only components at 20 Hz to 350 Hz, which are in a frequency band that the myoelectric potential waveform can take in the digital waveform signal, and remove components at frequencies lower than 20 Hz and components at frequencies higher than 350 Hz.

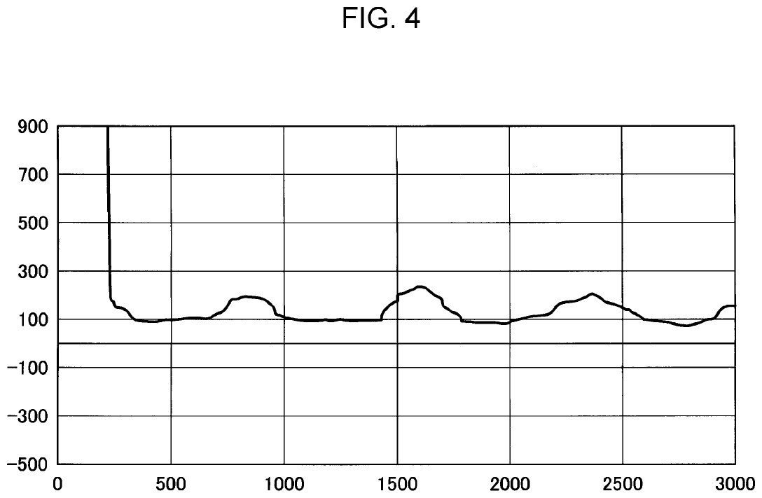

FIG. 4 illustrates a myoelectric potential waveform obtained when the waveform processing unit 122 performs waveform processing on the component waveform illustrated in FIG. 3. When waveform processing is performed to convert the amplitude of the component waveform illustrated in FIG. 3 so as to create a signal waveform that has root-mean-square (RMS) values as the amplitude values, the myoelectric potential waveform illustrated in FIG. 4 is obtained. The myoelectric potential waveform is obtained by squaring the positive component and negative component of the component waveform illustrated in FIG. 3 and calculating a square root of them.

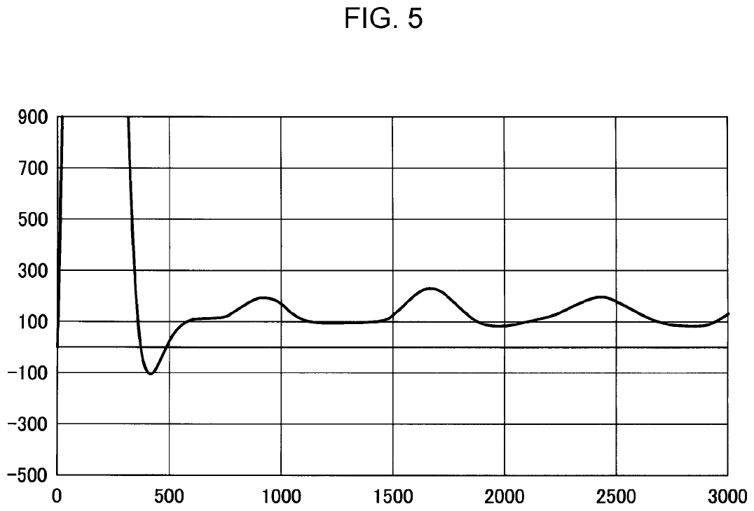

FIG. 5 illustrates a myoelectric potential waveform that the waveform processing unit 122 obtains by performing low-pass filter processing on the myoelectric potential waveform illustrated in FIG. 4. It is said that the number of revolutions per minute when a human pedals a bicycle is 200 revolutions per minute (rpm), which is about 3.35 Hz, or less. Therefore, components at 3.35 Hz or lower are extracted from the myoelectric potential waveform illustrated in FIG. 4, by performing low-pass filter processing in which 3.35 Hz is used as the cut-off frequency.

FIG. 6 is a drawing used to explain processing executed by the cadence deriving unit 123. In FIG. 6, the period T is a three-second period in the myoelectric potential waveform. The reason why the period T is set to three seconds as an example is that the period T is thought to include a plurality of cycles during which the user pedals.

The cadence deriving unit 123 obtains the average value A of amplitude data in the period T, and divides the period T into a plurality of cycles with the amplitude center of the myoelectric potential waveform in the period T taken as the average value A. In FIG. 6, three cycles denoted T1, T2, and T3 are included in the period T. There is a residual, which is shorter than one cycle, before cycle T1. The cadence deriving unit 123 obtains cycles T1, T2, and T3.

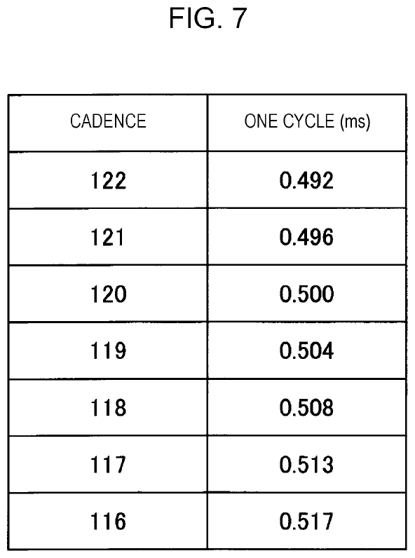

FIG. 7 is drawing indicating table data that relates cadence and the period (in milliseconds (ms)) of one cycle to each other. The table data in FIG. 7 relates the period of one revolution (period of one cycle) and cadence used as the number of revolutions of the crank to each other. As an example, FIG. 7 illustrates the period (0.517 ms to 0.492 ms) of one cycle when cadence is 116 to 122 (revolutions/minute).

The cadence deriving unit 123 references the table data and derives the cadence corresponding to the period of one cycle closest to any one of the periods of cycles T1 to T3. As a result, it will be assumed as an example that the cadence deriving unit 123 has derived that cadence in period T1 is 120 (revolutions/minute), cadence in period T2 is 119 (revolutions/minute), and cadence in period T3 is 118 (revolutions/minute). The cadence measurement device 100 obtains cadence from the myoelectric potential waveform in the way described above.

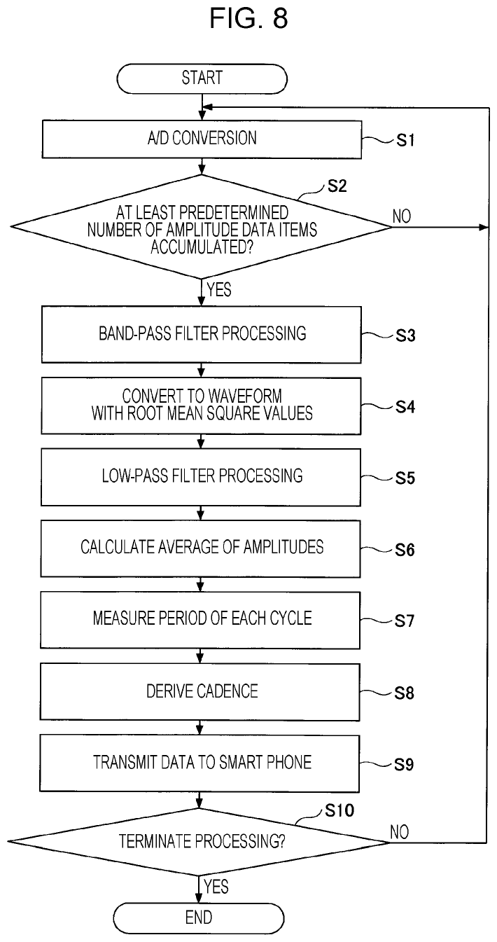

FIG. 8 is a flowchart illustrating processing executed by the cadence measurement device 100. Now, it will be assumed that the cadence measurement device 100 has been stuck to the thigh of the user, the power supply of the cadence measurement device 100 is turned on, and the myoelectric potential sensor 110 is performing measurement of the myoelectric potential. Processing illustrated in FIG. 8 is implemented by the cadence measurement method in an embodiment.

The main control unit 121 performs A/D conversion processing on the output signal from the myoelectric potential sensor 110 to convert the output signal to a digital waveform signal (step S1). Specifically, as an example, to create a digital waveform signal, the main control unit 121 performs A/D conversion on the output signal from the myoelectric potential sensor 110 at a sampling cycle of 0.1 ms, after which the main control unit 121 stores amplitude data of the digital waveform signal in the memory 124.

The waveform processing unit 122 decides whether at least a predetermined number of amplitude data items of the digital waveform signal have been accumulated (step S2). Here, at least the predetermined number of amplitude data items of the digital waveform signal refer to amplitude data items of the digital waveform signal for three seconds or more, as an example. This is because the periods of a plurality of cycles included in the period T for three seconds will be obtained later as in FIG. 6.

If the waveform processing unit 122 decides that at least the predetermined number of amplitude data items of the digital waveform signal have been accumulated (YES in S2), the waveform processing unit 122 performs band-pass filter processing on the digital waveform signals (step S3). Thus, component waveforms after only components at 20 Hz to 350 Hz, which are in a frequency band that the myoelectric potential waveform can take, in the digital waveform signal has been passed are obtained as illustrated in FIG. 3. If the waveform processing unit 122 decides in step S2 that at least the predetermined number of amplitude data items of the digital waveform signal have not been accumulated (No in S2), the waveform processing unit 122 returns the flow to step S1. This is because the waveform processing unit 122 waits for amplitude data to be further accumulated.

The waveform processing unit 122 performs waveform processing to convert the amplitude of the component waveform so as to create a signal waveform that has root-mean-square (RMS) values as the amplitude values (step S4). As a result, a myoelectric potential waveform as illustrated in FIG. 4 is obtained.

The waveform processing unit 122 performs low-pass filter processing on the myoelectric potential waveform (step S5). As a result, components at 3.35 Hz or lower are extracted from the myoelectric potential waveforms obtained in step S4, and a myoelectric potential waveform as illustrated in FIG. 5 is obtained.

The cadence deriving unit 123 calculates the average value A of amplitude data in the period T (step S6). The period T (see FIG. 6) is a period that includes at least the predetermined number of amplitude data items of the digital waveform signal, which have been obtained in step S2. That is, the period T is determined in step S2.

The cadence deriving unit 123 measures the period of each cycle included in the period T (step S7). As an example, processing in step S7 is to measure the periods of a plurality of cycles T1, T2, and T3 included in the period T, as described with reference to FIG. 6. To measure the periods of cycles T1, T2, and T3, it is only necessary to obtain an interval between sampling points, at which the amplitude data of the myoelectric potential waveform is the same as the average value A, in the direction of time. In this case, if there is no sampling point, at which a match is found between the amplitude data and the average value A, in the myoelectric potential waveform, interpolation or the like can be performed for two sampling points between which the average value A is present.

The cadence deriving unit 123 references the table data and derives the cadence corresponding to the period of one cycle closest to any one of the periods of cycles T1 to T3 (step S8).

The main control unit 121 causes the communication unit 130 to transmit data representing cadence to the smart phone 10 (step S9). Then, the smart phone 10 acquires the data representing cadence and displays the data on the display. The user can know the cadence by seeing the display of the smart phone 10.

The main control unit 121 decides whether to terminate the processing (step S10). The time to terminate the processing is when a manipulation to terminate measurement processing by the cadence measurement device 100 is performed. If the main control unit 121 decides that the processing is not to be terminated (NO in S10), the main control unit 121 returns the flow to step S1. If the main control unit 121 decides that the processing is to be terminated (YES in S10), the main control unit 121 terminates a series of processing.

As described above, the cadence measurement device 100 obtains a myoelectric potential waveform by performing filter processing on an output signal representing the myoelectric potential detected by the myoelectric potential sensor 110 and performing waveform processing such as processing to covert to root mean square values, after which the cadence measurement device 100 derives cadence from cycles included in the myoelectric potential waveform. In this case, there is no need to use either a rotational speed sensor or an acceleration sensor and the like, unlike the past. Therefore, the cadence measurement device 100 can be implemented with a very simple structure.

Therefore, it is possible to provide the cadence measurement device 100 having a simple structure and the cadence measurement method. Since the cadence measurement device 100 can be implemented only with the myoelectric potential sensor 110, MCU 120, communication unit 130, and battery 140, the cadence measurement device 100 is very small and lightweight. Therefore, even when the user sticks the cadence measurement device 100 to the thigh with a tape or the like, the cadence measurement device 100 is less likely to drop off and stable measurement can be continued for a long period of time. Also, the cadence measurement device 100 stuck to the thigh of the user produces only very little feeling of discomfort, so the cadence measurement device 100 is superior in ease of attachment.

In processing performed by an application program in the smart phone 10 after the myoelectric potential waveform measured by the cadence measurement device 100 is transmitted to the smart phone 10, the degree of fatigue of muscles of the user and the like may be inferred and the inferred result may be displayed on the display together with cadence. Then, the user can quantitatively grasp the degree of fatigue of muscles of the user together with the cadence.

An aspect has been described above in which the cadence deriving unit 123 measures the period of each cycle included in the period T and derives the cadence corresponding to the period of one cycle closest to any one of periods of cycles T1 to T3. However, table data that relates cadence and the number of revolutions of the crank per second to each other may have been stored in the memory 124 instead of the table data illustrated in FIG. 7. Then, in the table data, the cadence closest to the reciprocal (number of revolutions of the crank per second) of the period of each cycle may be derived, the reciprocal being obtained by performing a Fourier transform on the myoelectric potential waveform illustrated in FIG. 5.

This completes the description of the cadence measurement device and cadence measurement method in exemplary embodiments in the present invention. However, the present invention is not limited to specifically disclosed embodiments, but can be varied and modified in various ways without departing from the scope of the claims.

This international application claims priority based on Japanese Patent Application No. 2020-082718 filed on May 8, 2020, and the entire contents of the application are incorporated in this international application by reference.

What is claimed is:

1. A cadence measurement device, comprising:
a myoelectric potential sensor attachable to a leg portion of a living body;
a waveform processing unit that performs waveform processing to obtain a myoelectric potential waveform from an output signal from the myoelectric potential sensor; and
a cadence deriving unit that derives, when an average value of an amplitude in a plurality of cycles of the myoelectric potential waveform obtained by the waveform processing is taken as an amplitude center and the myoelectric potential waveform is divided into each cycle according to the amplitude center, cadence according to a period of each cycle of the plurality of cycles.

2. The cadence measurement device according to claim 1, wherein the waveform processing unit obtains the myoelectric potential waveform from the output signal by performing, as the waveform processing, processing to covert the output signal from the myoelectric potential sensor to a signal waveform with a root mean square value.

3. The cadence measurement device according to claim 2, wherein the waveform processing unit obtains the myoelectric potential waveform from the output signal by performing, as the waveform processing, processing to extract a component waveform, which is a waveform of a frequency component included in the myoelectric potential waveform of the living body, from the output signal from the myoelectric potential sensor and processing to convert the component waveform to a signal waveform with a root mean square value.

4. The cadence measurement device according to claim 1, further comprising a memory that stores table data that relates a number of crank revolutions and a period of one cycle, wherein
the cadence deriving unit derives, as the cadence, the number of crank revolutions that corresponds, in the table data, to the period of each cycle.

5. A cadence measurement method comprising:
performing waveform processing to obtain a myoelectric potential waveform from an output signal from a myoelectric potential sensor attached to a leg portion of a living body; and
deriving, when an average value of an amplitude in a plurality of cycles of the myoelectric potential waveform obtained by the waveform processing is taken as an amplitude center and the myoelectric potential waveform is divided into each cycle according to the amplitude center, cadence according to a period of each cycle of the plurality of cycles.

6. The cadence measurement method according to claim 5, wherein the waveform processing obtains the myoelectric potential waveform from the output signal by performing, as the waveform processing, processing to covert the output signal from the myoelectric potential sensor to a signal waveform with a root mean square value.

7. The cadence measurement method according to claim 6, wherein the waveform processing obtains the myoelectric potential waveform from the output signal by performing, as the waveform processing, processing to extract a component waveform, which is a waveform of a frequency component included in the myoelectric potential waveform of the living body, from the output signal from the myoelectric potential sensor and processing to convert the component waveform to a signal waveform with a root mean square value.

8. The cadence measurement method according to claim 5, further comprising storing in a memory table data that relates a number of crank revolutions and a period of one cycle, wherein
deriving, as the cadence, the number of crank revolutions that corresponds, in the table data, to the period of each cycle.
